(19) United States
(12) Patent Application Publication
(10) Pub. No.: US 2019/0201660 A1
(43) Pub. Date: Jul. 4, 2019

(54) Multi-Catheter Infusion System and Method of Use

(71) Applicant: Bloom Innovations LLC, Chicago, IL (US)

(72) Inventors: Neal Khurana, Chicago, IL (US); Osman Ahmed, Northbrook, IL (US)

(21) Appl. No.: 16/235,406

(22) Filed: Dec. 28, 2018

Related U.S. Application Data
(60) Provisional application No. 62/611,950, filed on Dec. 29, 2017.

(51) Int. Cl. A61M 25/00; A61M 25/01; A61M 25/06; A61M 25/09

(52) U.S. Cl. CPC A61M 25/003; A61M 25/0071; A61M 25/0133; A61M 25/005; A61M 25/09; A61M 2025/0037; A61M 25/0662

(57) ABSTRACT

The present disclosure relates generally to infusion catheters, and more particularly relates to a multi-catheter infusion system including a delivery sheath having a proximal end, a distal end, and a central axis. The delivery sheath includes a guidewire lumen extending therethrough that is aligned with the central axis within a head portion of the delivery sheath. First and second infusion lumens extend through the delivery sheath along respective paths from the proximal end to respective openings in the head portion. At the respective openings, the paths of the lumens are disposed at an acute angle to the central axis of the delivery sheath. First and second infusion catheters are disposed in the respective lumens and are movable relative to the delivery sheath so as to extend through the respective openings and transition from retracted positions to extended positions relative to the distal end of the delivery sheath.

### CROSS REFERENCE TO RELATED APPLICATIONS

This application claims the benefit of U.S. Provisional Application No. 62/611,950, filed Dec. 29, 2017, the disclosure of which is hereby incorporated by reference in its entirety.

### BACKGROUND OF THE DISCLOSURE

### 1. Field of the Disclosure

The present disclosure relates generally to infusion catheters for delivering therapeutic agents, for example, suitable for thrombolysis. The present disclosure relates more particularly to a multi-catheter system for delivering therapeutic agents to targeted areas.

### 2. Technical Background

Infusion catheters are used in a variety of different surgical applications. For example, a catheter may be inserted into a patient's body, such as in a cavity, and a therapeutic agent may be administered locally to the location of the catheter. In order to increase the area where the therapeutic agents are delivered, some infusion catheters include a number of openings where the agent can be released from the catheter. The ability of this construction to reach different areas that need treatment can be limited, however, particularly if reaching the different areas would require the catheter to travel in different directions within the patient's body.

In thrombolysis, for example, a catheter can be inserted into a patient's body in order to deliver a thrombolytic agent, such as tissue plasminogen activator, in the vicinity of a clot, or thrombus, within the patient's vasculature. This kind of therapy can be very effective at reducing or eliminating clots in the patient's blood vessels, which can prevent heart attacks or other cardiovascular conditions.

If the patient has multiple clots, however, treating the clots becomes more difficult. In particular, if there are clots in different blood vessels, treating all of the thrombus simultaneously with a single catheter can be impossible.

Further, if the thrombolytic agent is dispensed only in a vicinity of the various thrombus, it can dissipate quickly through unobstructed blood vessels, rather than effectively treating the thrombus. Alternatively, each thrombus can be treated separately. Although this method is effective, it can be very slow and costly.

Therefore, the inventors have determined that a catheter infusion system that can simultaneously target different blood vessels would be very attractive to the medical community.

### SUMMARY OF THE DISCLOSURE

In one aspect, the present disclosure provides a multi-catheter infusion system comprising:

a delivery sheath including:
a proximal end, a distal end, and a central axis,
a head portion disposed at the distal end of the delivery sheath;
a guidewire lumen extending through the delivery sheath and configured to receive a guidewire for positioning the delivery sheath, the guidewire lumen being aligned with the central axis of the delivery sheath within the head portion,
a first infusion lumen extending through the delivery sheath along a first path from the proximal end to a first opening in the head portion, wherein the first path, at the first opening, is disposed at an acute angle to the central axis of the delivery sheath, and
a second infusion lumen extending through the delivery sheath along a second path from the proximal end to a second opening in the head portion, wherein the second path, at the second opening, is disposed at an acute angle to the central axis of the delivery sheath.

In another aspect, the disclosure provides a method of delivering a therapeutic agent through a multi-catheter infusion system, the method comprising:

providing a delivery sheath including:
a proximal end, a distal end, and a central axis,
a head portion disposed at the distal end of the delivery sheath,
a guidewire lumen that extends through the delivery sheath and is aligned with the central axis of the delivery sheath within the head portion,
a first infusion lumen extending through the delivery sheath along a first path from the proximal end to a first opening in the head portion, and
a second infusion lumen extending through the delivery sheath along a second path from the proximal end to a second opening in the head portion,

advancing a first infusion catheter through the first infusion lumen so as to extend through the first opening at an acute angle to the central axis of the delivery sheath;

delivering a first therapeutic agent through the first infusion catheter;

advancing a second infusion catheter through the second infusion lumen so as to extend through the second opening at an acute angle to the central axis of the delivery sheath; and

delivering a second therapeutic agent through the second infusion catheter.

Additional aspects of the disclosure will be evident from the disclosure herein.

### BRIEF DESCRIPTION OF THE DRAWINGS

The accompanying drawings are included to provide a further understanding of the methods and devices of the disclosure, and are incorporated in and constitute a part of this specification. The drawings are not necessarily to scale, and sizes of various elements may be distorted for clarity. The drawings illustrate one or more embodiment(s) of the disclosure, and together with the description serve to explain the principles and operation of the disclosure.

FIG. 1 is a schematic side view of a delivery sheath of a multi-catheter infusion system according to an embodiment of the disclosure;

FIG. 2 is a schematic perspective view of a proximal end of a delivery sheath of the system in FIG. 1;

FIG. 3 is a schematic cross-sectional view of the delivery sheath of FIG. 1;

FIG. 4 is a schematic cross-sectional view of the delivery sheath of FIG. 1 including the guidewire and two infusion catheters;

FIG. 5 is a schematic perspective view of a distal end of the delivery sheath of the system of FIG. 1;

FIG. 6 is a schematic side view of the distal end of the delivery sheath of FIG. 1, with infusion catheters in a retracted position;

FIG. 7 is a schematic side view of the distal end of the delivery sheath of FIG. 1 with the infusion catheters in an extended position;

FIG. 8 is a flow chart illustrating a method according to an embodiment of the disclosure; and

FIG. 9 is a schematic cross section of the system of FIG. 1 in use during a method of the disclosure.

### DETAILED DESCRIPTION

As described above, the present inventors have noted that conventional infusion catheter systems are inefficient for providing treatment in different locations, such as in different blood vessels.

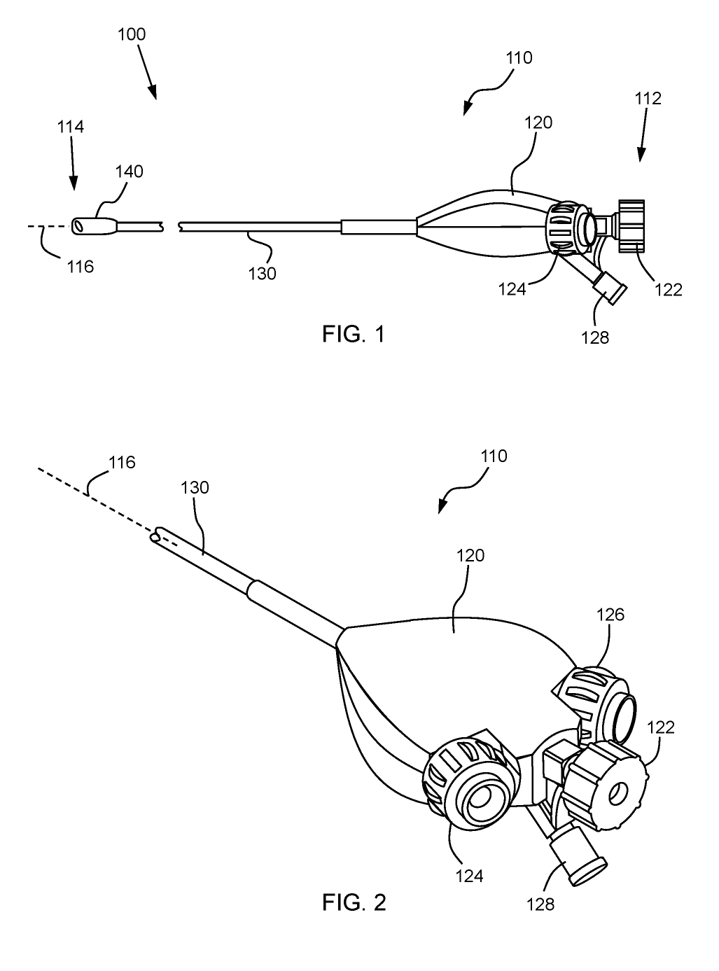

Accordingly, one aspect of the disclosure is a multi-catheter infusion system including a delivery sheath having a proximal end, a distal end, and a central axis. The delivery sheath includes a head portion disposed at the distal end of the delivery sheath and a guidewire lumen extending through the delivery sheath. The guidewire lumen is configured to receive a guidewire for positioning the delivery sheath. Further, within the head portion, the guidewire lumen is aligned with the central axis of the delivery sheath. A first infusion lumen extends through the delivery sheath along a first path from the proximal end to a first opening in the head portion. The first path, at the first opening, is disposed at an acute angle to the central axis of the delivery sheath. A second infusion lumen extends through the delivery sheath along a second path from the proximal end to a second opening in the head portion. The second path, at the second opening, is disposed at an acute angle to the central axis of the delivery sheath. Such a multi-catheter infusion system 100, is shown in FIGS. 1 through 7, and shown in use in FIG. 9.

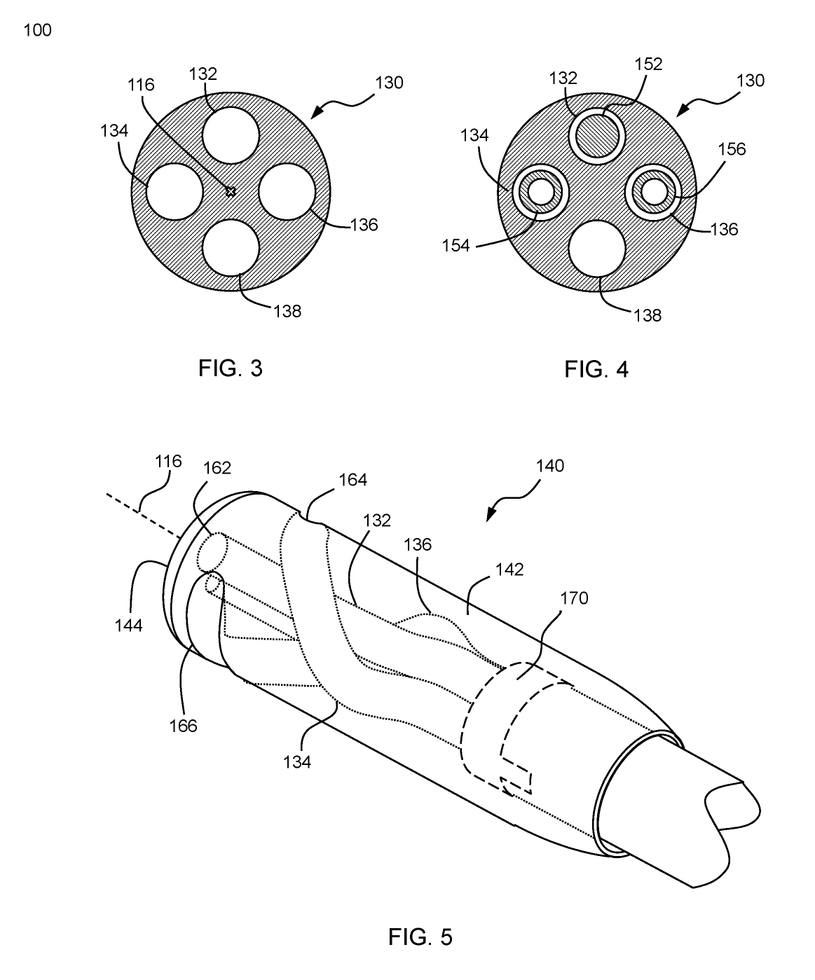

Multi-catheter infusion system 100 includes a delivery sheath 110 having a base 120 disposed at a proximal end 112 of the delivery sheath. Base 120 includes various ports 122, 124, 126 and 128. Each of the ports is configured to receive a wire, catheter and/or a substance that is provided through delivery sheath 110, as discussed in further detail below. An elongate sheath body 130 extends from base 120 along a central axis 116 to a distal end 114 of the delivery sheath 110 and includes a plurality of lumens 132, 134, 136, 138, as shown in the cross-sectional view of sheath body 130 presented in FIG. 3. In particular, in this example embodiment, the plurality of lumens includes a guidewire lumen 132 as well as first and second infusion lumens 134, 136. A central axis 116 of delivery sheath 110, as described herein, is defined with respect to a cross section of the elongate delivery sheath 110.

A head portion 140 is disposed at distal end 114 of delivery sheath 110 and includes an outer side surface 142 and a distal tip 144. A detailed view of head portion 140 is shown in FIG. 5. Head portion 140 also includes a plurality of openings that are in fluid communication with the lumens 132 through 138 that extend through delivery sheath 110. Head portion 140 of delivery sheath 110 is formed as a cap and has a bulbous shape with respect to sheath body 130. In other embodiments, the head portion is provided by the end portion of the sheath body, without a cap or any other added structure.

Guidewire lumen 132 is configured to receive a guidewire that is operable to position the delivery sheath, for example, inside a patient. Within head portion 140, guidewire lumen 132 is aligned with central axis 116. In particular, guidewire lumen 132 is aligned with central axis 116 at distal end 114 of delivery sheath 110, where guidewire lumen 132 includes a guidewire opening 162. The term “aligned,” as used herein, refers to an overlap between the lumen and the central axis, such that the central axis runs through the lumen. In particular, central axis 116 is surrounded by the outer wall of guidewire lumen 132. A guidewire 152 is shown within guidewire lumen 132 in the cross section shown in FIG. 4. In some embodiments, the guidewire lumen is coaxial with the central axis, such that the central axis is at the center of the guidewire lumen within the head portion, while in other embodiments the guidewire lumen is offset from the central axis.

While guidewire lumen 132 is aligned with central axis 116 within head portion 140, guidewire lumen 132 is not aligned with central axis 116 along the entire length of delivery sheath 110. Specifically, within elongate sheath body 130, guidewire lumen 132 runs parallel to central axis 116 and is offset therefrom, as illustrated in FIG. 3. Within head portion 140, guidewire lumen 132 shifts to align with central axis 116 as guidewire lumen 132 reaches opening 162. In contrast, in other embodiments, guidewire lumen is aligned with, or even coaxial with the central axis along the entire length of the head portion and sheath body. And in other embodiments, the guidewire lumen is offset from the central axis for the entire length of the head portion and sheath body.

Delivery sheath 110 also includes first and second infusion lumens 134, 136 that extend along respective paths through the delivery sheath from base 120 at proximal end 112 to head portion 140 at distal end 114. Each of infusion lumens 134, 136 are provided with a respective opening 164, 166 in head portion 140. While infusion lumens 134, 136 run straight through elongate body 130 of delivery sheath 110, as illustrated by FIG. 3, the respective paths of lumens 134, 136 are at least partially curved within head portion 140. The respective curves of the lumen paths within head portion 140 assist in directing objects or substances exiting the lumens in a predetermined direction. In particular, in head portion 140, the respective paths of first and second infusion lumens 134, 136 are angled such that, at openings 164, 166 of the lumens, the paths are at an acute angle to central axis 116 of delivery sheath 110.

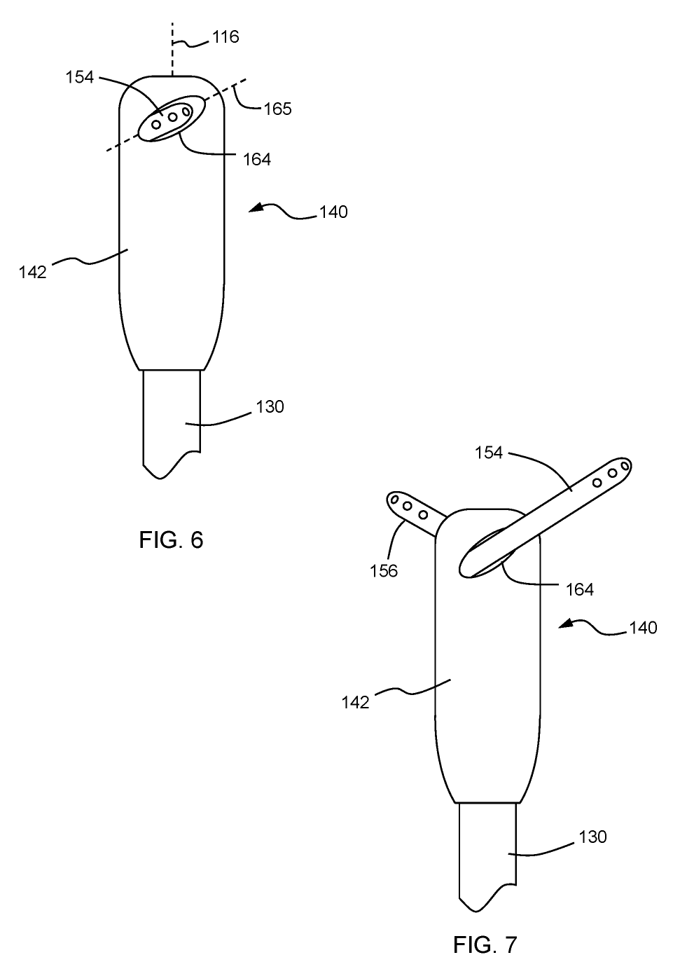

In example embodiments of the multi-catheter infusion system described herein, the system further includes a first infusion catheter that is disposed in the first infusion lumen and is movable relative to the delivery sheath so as to extend through the first opening and transition from a retracted position to an extended position relative to the distal end of the delivery sheath. The system also includes a second infusion catheter that is disposed in the second infusion lumen and is movable relative to the delivery sheath so as to extend through the second opening and transition from a retracted position to an extended position relative to the distal end of the delivery sheath. For example, system 100 includes first and second infusion catheters 154, 156 that are respectively disposed in first and second infusion lumens 134, 136. Each of the infusion catheters 154, 156 is independently movable relative to delivery sheath 110. Accordingly, infusion catheters 154, 156 can extend through the respective lumen openings 164, 166 in head portion 140 of the sheath. As a result, infusion catheters 154, 156 can transition from retracted positions, where the infusion catheters are shrouded by the delivery sheath, to extended positions relative to the distal end of delivery sheath 110. In other words, the distal end of each infusion catheter can independently extend out of the lumen opening in the head portion of the delivery sheath. FIGS. 6 and 7 illustrate infusion catheters 154, 156 in the retracted and extended positions, respectively. In FIG. 7, first infusion catheter 154 is extended further out of head portion 140 than second infusion catheter 156.

In example embodiments of the multi-catheter infusion system described herein, the guidewire may take a variety of different forms. In some embodiments, the guidewire includes a solid steel or nickel/titanium core. Further, in some embodiments, the core of the guidewire is wrapped in a coil or braid to enable suitable flexibility in positioning the tip of the guidewire in a desired location. Likewise, in other embodiments, the guidewire includes micro-cut slots to aid in flexibility. As will be appreciated by those of skill in the art, the pushability, steerability and torque of the guidewire may be selected in accordance with the use of the system. In some embodiments, the guidewire includes radiopaque markers, or a radiopaque wire component, to aid in navigating the guidewire during use.

In example embodiments of the multi-catheter infusion system described herein, the infusion catheters may take a variety of different forms. For example, in some embodiments, each infusion catheter includes a single opening at the distal tip of the catheter. In other embodiments, each infusion catheter includes a plurality of side holes. In some embodiments, the infusion catheters include a valved tip. Further, in some embodiments, the infusion catheters are infusion wires. Further still, in some embodiments, a combination of different infusion catheters including any of the above features may be used in the multi-catheter infusion system.

In example embodiments of the multi-catheter infusion system described herein, the delivery sheath further includes at least one additional infusion lumen extending through the delivery sheath to a respective additional opening in the head portion. For example, while delivery sheath 110 of multi-catheter system 100 includes two infusion lumens 134, 136, in some embodiments the delivery sheath of the disclosure includes three infusion lumens. In other embodiments, the delivery sheath includes four or more infusion lumens. As explained in more detail below, the use of multiple lumens allows for delivery of therapeutics through multiple catheters, which can be directed in order to target different areas for the delivery of the therapeutics. Thus, providing the delivery sheath with additional infusion catheters allows the system to supply therapeutics to more locations.

In example embodiments of the multi-catheter infusion system described herein, the guidewire lumen includes an opening at a distal tip of the head portion of the delivery sheath. For example, head portion 140 includes an opening 162 at the end of guidewire lumen 132. Opening 162 is specifically positioned on distal tip 144 of head portion 140. The inclusion of opening 162 at the distal end of sheath 110 allows a guidewire 152 to first be carefully positioned in a patient, and delivery sheath 110 to subsequently be advanced over the guidewire. In particular, the sheath can be advanced over the guidewire under fluoroscopic guidance.

In example embodiments of the multi-catheter infusion system described herein, the first path of the first infusion lumen includes a helical portion that extends at least partially around the guidewire lumen. For example, in delivery sheath 110 of system 100, the first path of infusion lumen 134 takes a helical path within head portion 140, partially extending around guidewire lumen 132 as it progresses toward opening 164 near distal tip 144. In particular, with respect to the orientation in FIG. 5, as first infusion lumen 134 extends toward distal tip 144, it helically curves from the near side of head portion 140 to the far side of head portion 140. Second infusion lumen 136 has a similar helical path and also partially wraps around guidewire lumen 132 within head portion 140. In some embodiments the infusion lumens have a longer helical path, extending entirely around the guidewire lumen at least once. In other embodiments, the infusion lumens do not have a helical path. For example, in some embodiments, the infusion lumens simply include a slight curve at their respective openings in the head portion of the delivery sheath in order to have a path that is disposed at an angle to the central axis of the delivery sheath.

In example embodiments of the multi-catheter infusion system described herein, the first path of the first infusion lumen, at the first opening, is not coplanar with the central axis. For example, at the location of first opening 164 in head portion 140, the path of first infusion lumen 132 is both offset from and at an angle to central axis 116. Accordingly, at opening 164, the path of first infusion lumen 132 and central axis 116 form skew lines that will not intersect. In other embodiments, the first infusion lumen curves away from the guidewire lumen so as to exit the head portion at an angle to the guidewire lumen. In such a case, the first infusion lumen and the guidewire lumen may be coplanar near the distal tip of the head portion.

In example embodiments of the multi-catheter infusion system described herein, the first opening forms an elliptical edge on an outer surface of the head portion of the delivery sheath. Further, in some embodiments, the semi-major axis of the elliptical edge is disposed at an angle to the central axis of the delivery sheath. For example, as shown in FIG. 6, first opening 164 at the end of first infusion lumen 134 has an elliptical edge at the outer side surface 142 of head portion 140. Further, the semi-major axis 165 of the elliptical edge of first opening 164 is disposed at an acute angle to central axis 116 of delivery sheath 110. The elliptical shape of opening 164 and the angle of its semi-major axis are described herein with reference to the outer side surface of the delivery sheath or the head portion of the delivery sheath. In some embodiments, the second opening at the end of the second infusion lumen also has an elliptical edge with a semi-major axis that is at an angle to the central axis of the delivery sheath. Still, in other embodiments, the opening at the end of one or more of the infusion lumens has another shape, such as a circular shape.

In example embodiments of the multi-catheter infusion system described herein, the shape of the opening at the end of the infusion lumens, and the direction of the path of the infusion lumens at the respective openings, may aid in positioning the corresponding infusion catheters. As a result, precise positioning of the infusion catheters may be possible without steering the individual infusion catheters themselves, which can allow the use of catheters that are less complicated and expensive. In other embodiments, the system uses one or more catheters that can be steered.

In example embodiments of the multi-catheter infusion system described herein, the delivery sheath further includes a contrast lumen including an opening at a distal tip of the head portion of the delivery sheath. For example, delivery sheath 110 includes a contrast lumen 138, as shown in FIG. 3. Contrast lumen 138 extends from base 120 to head portion 140 where it opens through distal tip 144. In other embodiments, the contrast lumen opens through a side surface of the head portion of the delivery sheath. Still in other embodiments, the delivery sheath does not include a contrast lumen.

In some embodiments of the multi-catheter system described herein, within the head portion, the contrast lumen has a smaller diameter than the guidewire lumen. For example, in system 100, contrast lumen 138 has a smaller diameter than guidewire lumen 132 within head portion 140, but has the same diameter as guidewire lumen 132 within the sheath body 130. In other embodiments, contrast lumen has a consistent diameter substantially along the entirety of the delivery sheath.

In some embodiments, the contrast lumen of the delivery sheath is configured to receive a contrast dye. The contrast dye may include a radiocontrast agent that is viewable using x-rays, fluoroscopy, or CT scans. The contrast dye may be beneficial during a surgical procedure by illuminating parts of the body that are of interest.

In example embodiments of the multi-catheter infusion system described herein, the delivery sheath includes a radiopaque marker at the distal end thereof. In some embodiments, the radiopaque marker is a marker band that encircles the head portion of the delivery sheath. For example, delivery sheath 110 includes a radiopaque marker band 170 within head portion 140. The radiopaque marker of the multi-catheter infusion system may take a variety of forms. For example, the marker may be platinum, a mixture of platinum and iridium, or gold. Various shapes and sizes of the marker are also possible, as will be appreciated by those of ordinary skill in the art. Further, as will be appreciated by those of ordinary skill in the art, the marker may be used to assist with positioning of the delivery sheath along the guidewire and, in some cases, past the end of the guidewire.

In example embodiments of the multi-catheter infusion system described herein, the base includes various ports for receiving catheters, wires or therapeutic substances. In some embodiments, the base includes at least one valve configured to provide a seal around an elongate object that is inserted through the valve, such as a hemostasis valve or a Touhy-Borst adapter. For example, base 120 of system 100 includes three fittings 122, 124, 126 that are adapted to receive wires and catheters while providing a tight seal around the wire/catheter. In some embodiments the base includes at least one fluid fitting in fluid communication with a lumen in the delivery sheath, such as a Luer tapered fitting. For example, base 120 of system 100 includes a Luer fitting 128 that is in fluid communication with contrast lumen 138, and is configured to receive a contrast dye.

In another aspect, the disclosure provides a method of delivering a therapeutic agent through a multi-catheter infusion system. The method includes providing a delivery sheath including a proximal end, a distal end, a central axis and a head portion disposed at the distal end of the delivery sheath. The delivery sheath also includes a guidewire lumen that extends through the delivery sheath and is aligned with the central axis of the delivery sheath within the head portion. A first lumen extends through the delivery sheath along a first path from the proximal end to a first opening in the head portion, and a second infusion lumen extends through the delivery sheath along a second path from the proximal end to a second opening in the head portion. The method further includes advancing a first infusion catheter through the first infusion lumen so as to extend through the first opening at an acute angle to the central axis of the delivery sheath, delivering a first therapeutic agent through the first infusion catheter, advancing a second infusion catheter through the second infusion lumen so as to extend through the second opening at an acute angle to the central axis of the delivery sheath, and delivering a second therapeutic agent through the second infusion catheter.

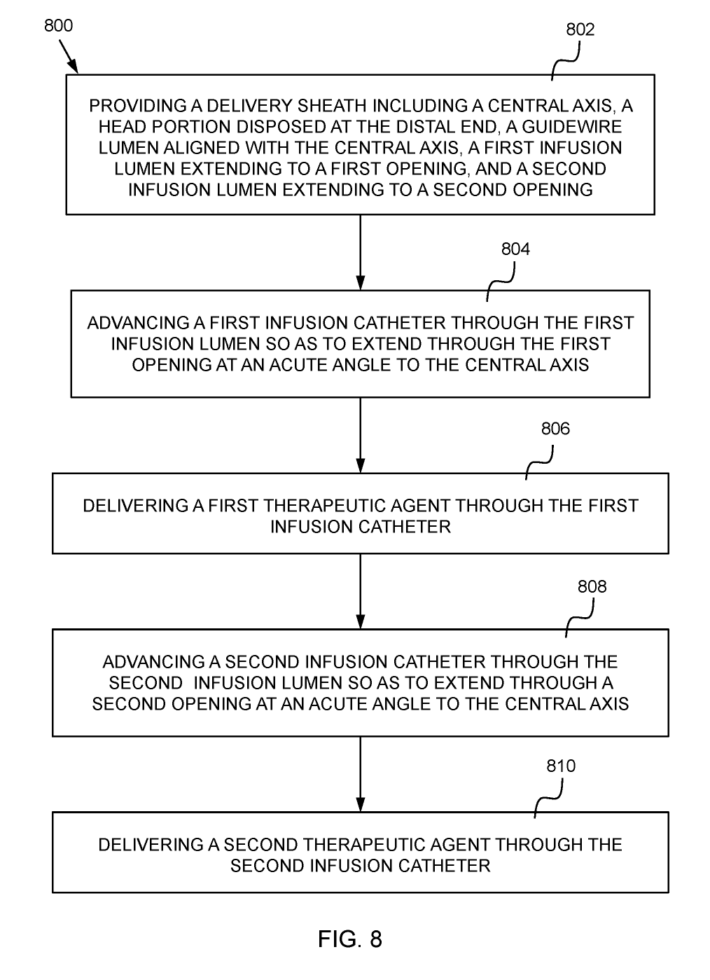

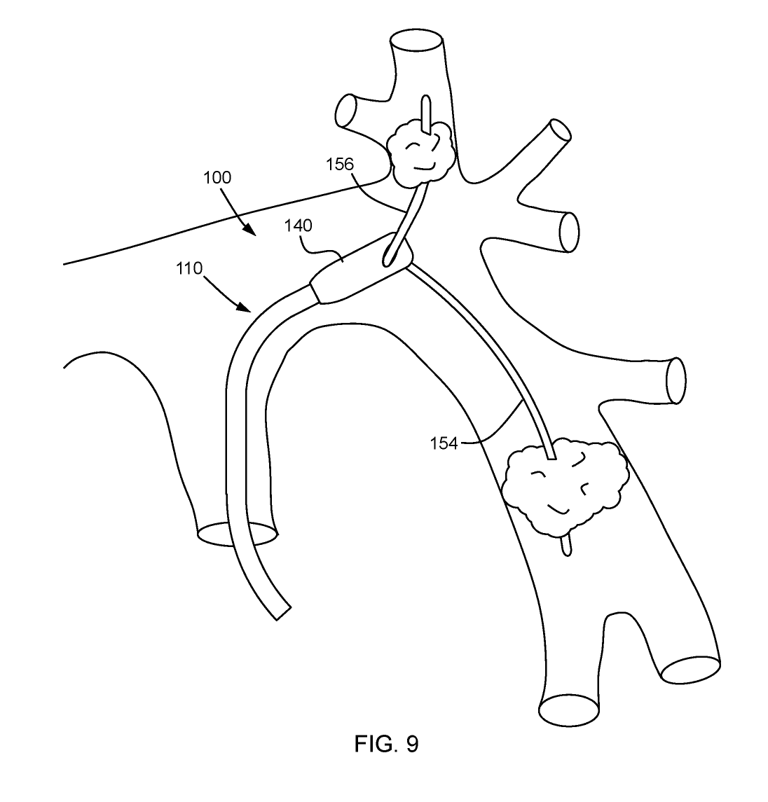

A flow chart illustrating such a method 800 of delivering a therapeutic agent through a multi-catheter infusion system is shown in FIG. 8. As shown by block 802, the method includes providing a delivery sheath including a central axis, a head portion disposed at the distal end of the delivery sheath, a guidewire lumen aligned with the central axis, a first infusion lumen extending to a first opening, and a second infusion lumen extending to a second opening. As shown by block 804, the method 800 further includes advancing a first infusion catheter through the first infusion lumen so as to extend through the first opening at an acute angle to the central axis. Further, as shown by block 806, the method includes delivering a first therapeutic agent through the first infusion catheter. The method similarly includes advancing a second infusion catheter through the second infusion lumen so as to extend through a second opening at an acute angle to the central axis, at block 808, and delivering a second therapeutic agent through the second infusion catheter, at block 810.

In example embodiments of the method described herein, the method further includes positioning the distal end of the delivery sheath in the vasculature of a person. In some embodiments, positioning the distal end of the delivery sheath in the vasculature of a person includes advancing the delivery sheath over a guidewire that extends through the guidewire lumen. For example, in some embodiments, the method includes inserting a guidewire into the vasculature of the patient, for example using internal jugular vein access or common femoral vein access. The guidewire is then advanced to a targeted region using an ultrasonic technique, as will be appreciated by those of ordinary skill in the art. In some embodiments, the delivery sheath of the multi-catheter system is then advanced over the guidewire under fluoroscopic guidance until the distal tip of the delivery sheath is in the target region. As examples, the system can be positioned in the main pulmonary artery, the right pulmonary artery, or the left pulmonary artery depending on the patient's needs, for example, the patient's thrombus burden and location. The result of such positioning is shown in FIG. 9. The delivery sheath 110 of multi-catheter infusion system 100 is shown in FIG. 9 as inserted into the vasculature of a patient, and the head portion 140 at the distal end of the delivery sheath is located near thrombus that are in need of treatment.

In some embodiments, once the delivery sheath is in the desired location, it is pinned in place outside of the patient's body. In some embodiments, the guidewire is removed after securing the delivery sheath. Removal of the guidewire may provide an additional lumen with access to the target region inside the patient's body.

In example embodiments of the method described herein, the first infusion catheter is advanced into a first blood vessel and the second infusion catheter is advanced into a second blood vessel that is different from the first blood vessel. In some embodiments, the first infusion catheter extends through the first opening further than the second infusion catheter extends through the second opening. For example, as shown in FIG. 9, first infusion catheter 154 extends from delivery sheath into a lower blood vessel and second infusion catheter 156 extends from delivery sheath 110 into an upper blood vessel that is different from the lower blood vessel. Further, first infusion catheter 154 extends further out of delivery sheath 110 than second infusion catheter 156. In other embodiments, the two infusion catheters are extended to the same distance out of the delivery sheath.

In example embodiments of the method described herein, a distal end of the first infusion catheter is inserted into a thrombus in the vasculature of the patient. Likewise, in some embodiments, the distal end of the second infusion catheter is inserted into another thrombus in the vasculature of the patient. For example, in FIG. 9, first infusion catheter 154 of system 100 is inserted into a thrombus in the lower vessel of the patient, while second infusion catheter is inserted into a different thrombus in the upper vessel of the patient. Here the terms “upper” and “lower” vessel are used only with respect to one another. This insertion of the infusion catheters into separate thrombus in different vessels allows for therapy that can address multiple locations simultaneously, reducing the time to treat the patient and the time that the patient's body must be in surgery, e.g., subject to x-ray radiation.

In example embodiments of the method described herein, the therapeutic agent is a thrombolytic agent. For example, in some embodiments, the therapeutic agent that is delivered through the infusion catheters is a lytic agent that breaks up the thrombus.

In example embodiments of the method described herein, the first infusion catheter and second infusion catheter are advanced in respective first and second directions by the angle of the respective openings of the first and second infusion lumens without additional guidance. For example, the angle of the first and second lumen paths at the opening where the infusion catheters exit the delivery sheath can be angled such that the infusion catheters are aimed in the desired direction. As such, in some embodiments, additional positioning and/or steering of the infusion catheters is not used. In other embodiments, the infusion catheters are individually positioned by steering or manipulating the distal end of the catheter.

In example embodiments of the method described herein, the first therapeutic agent and the second therapeutic agent are pumped through the respective first and second infusion catheters. For example, once the infusion catheters are located at the desired position, such as in a targeted thrombus, the first and second therapeutic agents can be delivered to the targeted areas by pumping the agents through the respective infusion catheters. In some embodiments, the first and second therapeutic agents have the same composition. In other embodiments, the first and second therapeutic agents are different. Likewise, in some embodiments, the first and second therapeutic agents are pumped in different quantities through each infusion catheter. This allows the medical practitioner to provide the proper quantity of agent to each targeted area, depending on the patient's needs and the thrombus burden in the various areas.

It will be apparent to those skilled in the art that various modifications and variations can be made to the processes and devices described here without departing from the scope of the disclosure. Thus, it is intended that the present disclosure cover such modifications and variations of this invention provided they come within the scope of the appended claims and their equivalents.

What is claimed is:

1. A multi-catheter infusion system comprising:
a delivery sheath including:
a proximal end, a distal end, and a central axis,
a head portion disposed at the distal end of the delivery sheath;
a guidewire lumen extending through the delivery sheath and configured to receive a guidewire for positioning the delivery sheath, the guidewire lumen being aligned with the central axis of the delivery sheath within the head portion,
a first infusion lumen extending through the delivery sheath along a first path from the proximal end to a first opening in the head portion, wherein the first path, at the first opening, is disposed at an acute angle to the central axis of the delivery sheath, and
a second infusion lumen extending through the delivery sheath along a second path from the proximal end to a second opening in the head portion, wherein the second path, at the second opening, is disposed at an acute angle to the central axis of the delivery sheath.

2. The multi-catheter infusion system of claim 1, further comprising:
a first infusion catheter disposed in the first infusion lumen and movable relative to the delivery sheath so as to extend through the first opening and transition from a retracted position to an extended position relative to the distal end of the delivery sheath; and
a second infusion catheter disposed in the second infusion lumen and movable relative to the delivery sheath so as to extend through the second opening and transition from a retracted position to an extended position relative to the distal end of the delivery sheath.

3. The multi-catheter infusion system of claim 1, wherein the delivery sheath further includes at least one additional infusion lumen extending through the delivery sheath to a respective additional opening in the head portion.

4. The multi-catheter infusion system of claim 1, wherein the guidewire lumen includes an opening at a distal tip of the head portion of the delivery sheath.

5. The multi-catheter infusion system of claim 1, wherein the first path of the first infusion lumen includes a helical portion that extends at least partially around the guidewire lumen.

6. The multi-catheter infusion system of claim 5, wherein the first path of the first infusion lumen, at the first opening, is not coplanar with the central axis.

7. The multi-catheter infusion system of claim 5, wherein the first opening forms an elliptical edge on an outer surface of the head portion of the delivery sheath.

8. The multi-catheter infusion system of claim 7, wherein the semi-major axis of the elliptical edge is disposed at an angle to the central axis.

9. The multi-catheter infusion system of claim 1, wherein the delivery sheath further includes a contrast lumen including an opening at a distal tip of the head portion of the delivery sheath.

10. The multi-catheter infusion system of claim 1, wherein the delivery sheath includes a radiopaque marker at the distal end thereof.

11. The multi-catheter infusion system of claim 10, wherein the radiopaque marker is a marker band that encircles the head portion of the delivery sheath.

12. A method of delivering a therapeutic agent through a multi-catheter infusion system, the method comprising:
providing a delivery sheath including:
a proximal end, a distal end, and a central axis,
a head portion disposed at the distal end of the delivery sheath,
a guidewire lumen that extends through the delivery sheath and is aligned with the central axis of the delivery sheath within the head portion,
a first infusion lumen extending through the delivery sheath along a first path from the proximal end to a first opening in the head portion, and
a second infusion lumen extending through the delivery sheath along a second path from the proximal end to a second opening in the head portion,
advancing a first infusion catheter through the first infusion lumen so as to extend through the first opening at an acute angle to the central axis of the delivery sheath;
delivering a first therapeutic agent through the first infusion catheter;
advancing a second infusion catheter through the second infusion lumen so as to extend through the second opening at an acute angle to the central axis of the delivery sheath; and
delivering a second therapeutic agent through the second infusion catheter.

13. The method of claim 12, further comprising positioning the distal end of the delivery sheath in the vasculature of a person.

14. The method of claim 13, wherein positioning the distal end of the delivery sheath in the vasculature of a person includes advancing the delivery sheath over a guidewire that extends through the guidewire lumen.

15. The method of claim 13, wherein the first infusion catheter is advanced into a first blood vessel and the second infusion catheter is advanced into a second blood vessel that is different from the first blood vessel.

16. The method of claim 15, wherein the first infusion catheter extends through the first opening further than the second infusion catheter extends through the second opening.

17. The method of claim 13, wherein a distal end of the first infusion catheter is inserted into a thrombus in the vasculature of the patient.

18. The method of claim 17, wherein the therapeutic agent is a thrombolytic agent.

19. The method of claim 13, wherein the first infusion catheter and second infusion catheter are advanced in respective first and second directions by the angle of the respective openings of the first and second infusion lumens without additional guidance.

20. The method of claim 12, wherein the first therapeutic agent and the second therapeutic agent are pumped through the respective first and second infusion catheters.
